(19) United States
(12) Patent Application Publication
(10) Pub. No.: US 2005/0202402 A1
(43) Pub. Date: Sep. 15, 2005

(54) Tandem signaling assay

(75) Inventors: Cynthia C. Bamdad, Boston, MA (US); Shoshana Bamdad Shendelman, New York, NY (US)

Correspondence Address: Timothy J. Oyer, Ph.D.;Wolf, Greenfield & Sacks, P.C., 600 Atlantic Avenue, Boston, MA, 02210-2206, US

(73) Assignee: Minerva Biotechnologies Corporation, Waltham, MA (US)

(21) Appl. No.: 10/927,780

(22) Filed: Aug. 27, 2004

Related U.S. Application Data
(63) Continuation of application No. 10/694,525, filed on Oct. 27, 2003, now abandoned.
Application No. 10/694,525 is a continuation of application No. 10/325,670, filed on Dec. 20, 2002, now abandoned.
Application No. 10/325,670 is a continuation of application No. PCT/US01/19968, filed on Jun. 25, 2001.
(60) Provisional application No. 60/214,217, filed on Jun. 23, 2000.
(60) Provisional application No. 60/277,914, filed on Mar. 22, 2001.

(51) Int. Cl. C12Q 1/00; G01N 33/543

(52) U.S. Cl. 435/4; 436/523

(57) ABSTRACT

Chemical, biological, and/or biochemical assays of the invention utilize intermediate binding entities for binding interactions. For example, a colloid particle carrying a species that could harm a component in the assay is allowed to binding interact in the assay only after other, more sensitive components have finished their binding interactions. In another example, multi-step binding interactions are used where single-step interactions could be used, so as to reduce false positives.

### RELATED APPLICATIONS

This application is a continuation of U.S. Non-Provisional Patent Application Ser. No. 10/694,525, filed Oct. 27, 2003, entitled “Tandem Signaling Assay,” by Bamdad et al., which is a continuation of U.S. Non-Provisional Patent Application Ser. No. 10/325,670, filed Dec. 20, 2002, entitled “Tandem Signaling Assay,” by Bamdad, et al., which is a continuation of International Patent Application Serial No. PCT/US01/19968, filed Jun. 25, 2001, entitled “Tandem Signaling Assay,” by Bamdad, et al. which was published under PCT Article 21(2) in English, which application claims priority to U.S. Provisional Patent Application Ser. No. 60/214,217, filed Jun. 23, 2000, entitled “Tandem Signaling Assay,” by Bamdad, et al.; and U.S. Provisional Patent Application Ser. No. 60/277,914, filed Mar. 22, 2001, entitled “Tandem Signaling Assay,” by Bamdad, et al. The contents of each of these applications is incorporated herein by reference.

### FIELD OF THE INVENTION

This invention relates generally to chemical and biochemical detection methods, and more particularly to techniques in which colloids bind indirectly, e.g., through an intermediate entity, to non-colloidal structures such as electrodes, beads, cells, and the like.

### BACKGROUND OF THE INVENTION

It is important to study biological and chemical interactions such as specific biological binding. However, study of such interactions can be difficult when a particular species involved in an interaction to be studied either is inherently delicate or can be damaged in some way by a component, such as a signaling entity, used in the study. Studies involving cells can be particularly challenging.

Drug discovery is facilitated by screening large numbers of candidate compounds for interaction with target receptor or proteins under physiological conditions. Of particular importance are cell surface receptors or proteins. Many of the biomolecular interactions that promote tumorigenesis involve cell surface proteins that mediate both intra- and intercellular signaling. “Tumor markers” are molecules on the surface of a cell that are either exclusively expressed, or over-expressed, as a result of transformation to a neoplastic state. Some of these markers have been correlated with the ability of the tumor to invade tissues and display an aggressive course of growth characterized by metastases (these tumors generally are associated with a poor outcome for the patient). For example, the interaction between the cell surface receptor αVβ3 and the cell adhesion molecule vitronectin has been implicated in angiogenesis. See J. Varner et al., “Integrins and cancer,” Curr Opin Cell Biol. 8:724 (1996); B. Vailhe et al., “In vitro angiogenesis is modulated by the mechanical properties of fibrin gels and is related to a αVβ3 integrin localization,” In Vitro Cell Dev Biol Anim., 33:763 (1997); M. Horton, “The αVβ3 integrin ‘vitronectin receptor’,” Int J Biochem Cell Biol, 29:721 (1997). Indeed, the increased concentration of this receptor on a melanoma cell has been correlated with a poor prognosis. Another example of a cell surface receptor that promotes tumorigenesis and/or angiogenesis is the MUC-1 antigen; this antigen is overexpressed on breast, prostate, lung and ovarian cancers.

The search for drugs to bind to and block cell surface receptors implicated in tumorigenesis has been technically challenging because there are few assays that work with intact cells, as mentioned above. The ability to easily detect interactions between ligands and target receptors on the surface of live, intact cells, would enable the screening of candidate compounds to disrupt these interactions. Screening compound libraries for drugs that inhibit the action of cell surface receptors depends critically on the receptors being in their native conformation and multimerization state throughout the drug screening process. According to common current technologies, it is difficult or impossible to detect the interaction of cell surface receptors with their natural ligands.

Fluorescence activated cell sorting (FACS) is one of the few techniques that enables the detection of cell surface receptors. One complication in using this technique to screen for drugs to block cell surface receptors is that: 1) the technique is sequential and cannot be readily multiplexed to facilitate massive drug screening; and 2) the technique is only feasible for detecting antibodies bound to the receptors; antibody/antigen interactions are typically high affinity interactions that cannot be disrupted by drugs.

Another technique that can be used to query cell surface receptors is an ELISA assay. In this technique, cells are adhered to a 96-well plastic plate. A cognate antibody is allowed to bind to the cell surface receptor of interest and unbound antibody is washed away. The availability of the receptor is inferred by detecting the presence of the cognate antibody. The presence of bound antibody is detected by introducing a second antibody, conjugated to a detectable label, which is active against the species of the cognate antibody. There are several inherent problems which limit the usefulness of this technique for assessing the availability of cell surface proteins or for screening for drugs to block them: 1) the technique is plagued by false positives that result from the non-specific adsorption of antibodies to the plastic; 2) ligand-receptor interactions are often disrupted by the many washing steps; and most importantly, 3) antibodies rather than natural ligands must be used in this assay; because these are typically high affinity interactions, they cannot be readily disrupted by drugs that bind the receptor; additionally, unlike the natural ligand, the antibody may bind the target receptor at a site that does not disrupt the receptor's normal function.

What is needed is an approach for monitoring or controlling binding events between chemical or biological species that increases versatility and can increase sensitivity in a wide variety of specific interactions. A particularly useful system would allow the study of binding events using one or more components that can harm one or more other components in the event without such harm taking place.

### SUMMARY OF THE INVENTION

The present invention provides techniques for chemical and biological detection/determination.

In one embodiment, the invention provides a method allowing a colloid particle to become immobilized indirectly relative to a non-colloidal structure, and determining immobilization of the colloid particle relative to the non-colloidal structure.

In another embodiment, a method of the invention involves providing an intermediate entity carrying a first immobilized biological or chemical agent suspected of being a binding partner of a second biological or chemical agent. The second agent is immobilized relative to a non-colloidal structure. A colloid particle, having the known ability to become immobilized relative to the intermediate entity is provided, and the intermediate entity is exposed to the non-colloidal structure. The colloid particle is exposed to the intermediate entity. Binding of the first agent to the second agent is determined by determining immobilization of the colloid particle relative to the non-colloidal structure.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIG. 1 is a schematic illustration of determination of binding between a species immobilized with respect to a non-colloidal structure and a species immobilized with respect to an intermediate entity, and coupling of a signaling entity-carrying structure to the intermediate entity;

FIG. 2 schematically illustrates a cell at the surface of an electrode, intermediate entities carrying binding partners of cell surface receptors immobilized with respect to the cell, and signaling entity-carrying structures immobilized relative to the intermediate entities;

FIG. 3 illustrates a cell at a surface of an electrode, drugs bound to cell-surface receptors, and the intermediate entities and signaling structures of FIG. 2 unable to bind to the cell surface receptors;

FIG. 4 schematically illustrates a magnetic bead, magnetically drawn to the surface of an electrode, and carrying a first immobilized chemical or biological agent on its surface, intermediate entities immobilized relative to the magnetic bead via a second chemical or biological agent that is a binding partner of the first chemical or biological agent, and signaling structure immobilized relative to the intermediate entities; and

FIG. 5 schematically illustrates a cell immobilized relative to a magnetic bead and also immobilized relative to intermediate entities that are immobilized relative to signaling structures, all drawn to the surface of an electrode, magnetically.

### DETAILED DESCRIPTION OF THE INVENTION

International patent application serial number PCT/US00/01997, filed Jan. 25, 2000 by Bamdad et al., published Jul. 27, 2000 as International Patent Publication No. WO 00/43791, entitled “Rapid and Sensitive Detection of Aberrant Protein Aggregation in Neurodegenerative Diseases”; International patent application serial number PCT/US00/01504, filed Jan. 21, 2000 by Bamdad, et al, published Jul. 27, 2000 as International Patent Publication No. WO 00/43783, entitled “Interaction of Colloid-Immobilized Species with Species on Non-Colloidal Structures”; U.S. patent application Ser. No. 60/214,217, filed Jun. 23, 2000 by Bamdad, et al.; and U.S. patent application Ser. No. 60/277,914, filed Mar. 22, 2001 by Bamdad, et al., all are incorporated herein by reference.

### DEFINITIONS

“Small molecule”, as used herein, means a molecule less than 5 kiloDalton, more typically less than 1 kiloDalton. As used herein, “small molecule” excludes proteins.

The term “candidate drug” as used herein, refers to any medicinal substance used in humans, animals, or plants. Encompassed within this definition are compound analogs, naturally occurring, synthetic and recombinant pharmaceuticals, hormones, antimicrobials, neurotransmitters, etc. This includes any substance or precursor (whether naturally occurring, synthetic or recombinant) which is to be evaluated for use as a drug for treatment of neurodegenerative disease, or other disease characterized by aberrant aggregation, or prevention thereof. Evaluation typically takes place through activity in an assay, such as the screening assays of the present invention.

A variety of types of particles can be used in the invention. For example, “fluid-suspendable particle” means a particle that can be made to stay in suspension in a fluid in which it is used for purposes of the invention (typically an aqueous solution) by itself, or can be maintained in solution by application of a magnetic field, an electromagnetic field, agitation such as stirring, shaking, vibrating, sonicating, centrifuging, vortexing, or the like. A “magnetically suspendable” particle is one that can be maintained in suspension in a fluid via application of a magnetic field. An electromagnetically-suspendable particle is one that can be maintained in suspension in a fluid by application of an electromagnetic field (e.g., a particle carrying a charge, or a particle modified to carry a charge). A “self-suspendable particle” is a particle that is of low enough size and/or mass that it will remain in suspension in a fluid in which it is used (typically an aqueous solution), without assistance of for example a magnetic field, for at least 1 hour. Other self-suspendable particles will remain in suspension, without assistance, for 5 hours, 1 day, 1 week, or even 1 month, in accordance with the invention.

“Proteins” and “peptides” are well-known terms in the art, and are not precisely defined in the art in terms of the number of amino acids that each includes. As used herein, these terms are given their ordinary meaning in the art. Generally, peptides are amino acid sequences of less than about 100 amino acids in length, but can include sequences of up to 300 amino acids. Proteins generally are considered to be molecules of at least 100 amino acids.

As used herein, a “metal binding tag” refers to a group of molecules that can become fastened to a metal that is coordinated by a chelate. Suitable groups of such molecules include amino acid sequences including, but not limited to, histidines and cysteines (“polyamino acid tags”). Metal binding tags include histidine tags, defined below.

As used herein, “chelate coordinating a metal” or metal coordinated by a chelate, refers to a metal coordinated by a chelating agent that does not fill all available coordination sites on the metal, leaving some coordination sites available for binding via a metal binding tag. Exemplary chelates that coordinate a metal include nitrilotriacetic acid, 2,2′-bis(salicylideneamino)-6,6′-demethyldiphenyl, and 1,8-bis(a-pyridyl)-3,6-dithiaoctane, or the like.

As used herein, “metal binding tag/metal/chelate linkage” defines a linkage between first and second species in which a first species is immobilized relative to a metal binding tag and a second species is immobilized relative to a chelate coordinating a metal, where the chelate coordinates a metal to which the metal binding tag is also coordinated. U.S. Pat. No. 5,620,850 of Bamdad, et al., incorporated herein by reference, describes exemplary linkages.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., colloid particles), entities that absorb or emit electromagnetic radiation at a level or within a wavelength range such that they can be readily detected visibly (unaided or with a microscope including an electron microscope or the like), or spectroscopically, entities that can be detected electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition herein, phosphorescent moieties), up-regulating phosphors, chemiluminescent entities, electrochemiluminescent entities, or enzyme-linked signaling moieties including horse radish peroxidase and alkaline phosphatase. “Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein. See for example, Knight, “Trends in Analytical Chemistry”, vol. 18, 1999, pg. 47; Knight, et al., Analyst, vol. 119, 1994, page 879; Stults, et al., “Use of Recombinant Biotinylated Aequorin in Microtiter and Membrane-Based Assays: Purification of Recombinant Apoaequorin from escheria coli”, Biochemistry, 1992, 31, 1433; Mengeling, et al., “A Microplate Assay for Analysis for Solution-Phase Glycosyltransferase Reactions: Determination of Kinetic Constants”, Analytical Biochemistry, 119, 286, (1991). The invention can be particularly useful in connection with an electroactive signaling entity such as a metallocene, since metallocenes can be destructive of some components in some assays, such as cells. Metallocenes that can operate as electroactive signaling elements in accordance with the invention are known. One example of a particularly preferred signaling entity is one containing a ferrocene or a ferrocene derivative group or derivative, such as ferrocenyl thiol (C₃₅H₂₄FeS); however, other organic complexes of transitions metals are also contemplated as signaling elements. Metallocenes are particularly useful as signaling entities for the following reasons. Various ferrocene derivatives and be selected to each oxidize at unique voltage between 100 mV to 800 mV. Each oxidation potential represents a unique label so that multiple cell surface targets can be simultaneously queried. If a biologically relevant interaction between a cell surface receptor and a colloid immobilized ligand occurs, the cell is decorated with electronic or electrochemical signaling particles and a current peak results. The magnitude of the current peak should be proportional to the number of cell surface receptors that were recognized by the signaling colloids. See International Patent Application entitled, “Electroactive Surface-Confinable Molecules”, by Bamdad, et al., filed May 25, 2001 (International Pat. Apl. Ser. No. PCT/US01/40801, published on Dec. 6, 2001 as Publication No. WO 01/92277), and priority documents U.S. Provisional Application Ser. Nos. 60/207,387, filed May 26, 2000, and 60/277,861, filed Mar. 22, 2001, each incorporated herein by reference. Signaling entities can be easily be attached to intermediate entities such as colloids, polymers, etc. E.g. they can be attached to gold colloids that also present putative binding partners either through affinity tags, EDC/NHS chemistry or by binding to a His-tagged protein A or G presented on NTA-SAM-coated colloids according to the invention. Signaling entities such as fluorescent moieties also can be co-immobilized on a colloid via a biotin terminated ligand, or may be fastened via a chelate/metal/metal binding tag linkage. A fluorescent moiety may also be fastened by attaching it to an antibody and using a chelate/metal/metal binding tag with His-protein G to bind the antibody. The moieties can then be directly detected.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as protein A, which is covalently attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) which molecule also presents a chelate coordinating a metal. A species also is adapted to be fastened to a surface if a surface carries a particular nucleotide sequence, and the species includes a complementary nucleotide sequence.

“Covalently fastened” means fastened via nothing other than one or more covalent bonds. E.g. a species that is covalently coupled, via EDC/NHS chemistry, to a carboxylate-presenting alkyl thiol which is in turn fastened to a gold surface, is covalently fastened to that surface.

As used herein, a component that is “immobilized relative to” another component either is fastened to the other component or is indirectly fastened to the other component, e.g., by being fastened to a third component to which the other component also is fastened, or otherwise is translationally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc. All entities that can be fastened or adapted to be fastened to other entities of the invention also can be immobilized or adapted to be immobilized to the other entities, and vice versa.

“Specifically fastened” or “adapted to be specifically fastened” means a species is chemically or biochemically linked to another specimen or to a surface as described above with respect to the definition of “fastened to or adapted to be fastened”, but excluding all non-specific binding.

“Non-specific binding”, as used herein, is given its ordinary meaning in the field of biochemistry.

“Colloids”, as used herein, means nanoparticle, i.e. very small, self-suspendable particles including inorganic, polymeric, and metal particles. Typically, colloid particles are of less than 250 nm cross section in any dimension, more typically less than 100 nm cross section in any dimension, and preferably 10-30 nm, and can be metal, non-metal, crystalline or amorphous. As used herein this term includes the definition commonly used in the field of biochemistry, and it typically means gold colloid particles.

A “moiety that can coordinate a metal”, a used herein, means any molecule that can occupy at least two coordination sites on a metal atom, such as a metal binding tag or a chelate.

“Diverse biological species” means different animals, such as mouse and hamster, mouse and goat, etc.

The term “sample” refers to any cell, tissue, or fluid from a biological source (a “biological sample”), or any other medium, biological or non-biological, that can advantageously be evaluated in accordance with the invention including, but not limited to, a biological sample drawn from a human patient, a sample drawn from an animal, a sample drawn from food designed for human consumption, a sample including food designed for animal consumption such as livestock feed, milk, an organ donation sample, a sample of blood destined for a blood supply, a sample from a water supply, or the like. One example of a sample is a sample drawn from a human or animal to whom a candidate drug has been given to determine the efficacy of the drug.

A “structurally predetermined sample”, as used herein means samples, the chemical or biological sequence or structure of which is a predetermined structure used in an assay designed to test whether the structure is associated with a particular process such as a neurodegenerative disease. For example, a “structurally predetermined sample” includes a peptide sequence, random peptide sequence in a phage display library, and the like.

A “sample suspected of containing” a particular component means a sample with respect to which the content of the component is unknown. The sample may be unknown to contain the particular component, or may be known to contain the particular component but in an unknown quantity. For example, a fluid sample from a human suspected of having a disease, but not known to have the disease, defines a sample suspected of containing species uniquely associated with (indicative of) the disease.

As used herein, a “metal binding tag” refers to a group of molecules that can become fastened to a metal that is coordinated by a chelate. Suitable groups of such molecules include amino acid sequences, typically from about 2 to about 10 amino acid residues. These include, but are not limited to, histidines and cysteines (“polyamino acid tags”). Such binding tags, when they include histidine, can be referred to as a “poly-histidine tract” or “histidine tag” or “HIS-tag”, and can be present at either the amino- or carboxy-terminus, or at any exposed region, of a peptide or protein or nucleic acid. A poly-histidine tract of six to ten residues is preferred for use in the invention. The poly-histidine tract is also defined functionally as being a number of consecutive histidine residues added to a protein of interest which allows the affinity purification of the resulting protein on a metal chelate column, or the identification of a protein terminus through the interaction with another molecule (e.g. an antibody reactive with the HIS-tag).

“Affinity tag” is given its ordinary meaning in the art. Affinity tags include, for example, metal binding tags, GST (in GST/glutathione binding), and streptavidin (in biotin/streptavidin binding). At various locations herein specific affinity tags are described in connection with binding interactions. It is to be understood that the invention involves, in any embodiment employing an affinity tag, a series of individual embodiments each involving selection of any of the affinity tags described herein.

A “moiety that can coordinate a metal”, as used herein, means any molecule that can occupy at least two coordination sites on a metal atom, such as a metal binding tag or a chelate.

“Molecular wires” as used herein, means wires that enhance the ability for a fluid encountering a SAM-coated electrode to communicate electrically with the electrode. This includes conductive molecules or, as mentioned above and exemplified more fully below, molecules that can cause defects in the SAM allowing communication with the electrode. A non-limiting list of additional molecular wires includes 2-mercaptopyridine, 2-mercaptobenzothiazole, dithiothreitol, 1,2-benzenedithiol, 1,2-benzenedimethanethiol, benzene-ethanethiol, and 2-mercaptoethylether. Conductivity of a monolayer can also be enhanced by the addition of molecules that promote conductivity in the plane of the electrode. Conducting SAMs can be composed of, but are not limited to: 1) poly (ethynylphenyl) chains terminated with a sulfur; 2) an alkyl thiol terminated with a benzene ring; 3) an alkyl thiol terminated with a DNA base; 4) any sulfur terminated species that packs poorly into a monolayer; 5) all of the above plus or minus alkyl thiol spacer molecules terminated with either ethylene glycol units or methyl groups to inhibit non specific adsorption. Thiols are described because of their affinity for gold in ready formation of a SAM. Other molecules can be substituted for thiols as known in the art from U.S. Pat. No. 5,620,820, and other references. Molecular wires typically, because of their bulk or other conformation, creates defects in an otherwise relatively tightly-packed SAM to prevent the SAM from tightly sealing the surface against fluids to which it is exposed. The molecular wire causes disruption of the tightly-packed self-assembled structure, thereby defining defects that allow fluid to which the surface is exposed to communicate electrically with the surface. In this context, the fluid communicates electrically with the surface by contacting the surface or coming in close enough proximity to the surface that electronic communication via tunneling or the like, can occur.

The term “binding partner” refers to a molecule that can undergo binding with a particular molecule. Biological binding partners are examples. For example, Protein A is a binding partner of the biological molecule IgG, and vice versa.

The term “biological binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules such as biological binding partners including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “determining” refers to quantitative or qualitative analysis of a species via, for example, spectroscopy, ellipsometry, piezoelectric measurement, immunoassay, electrochemical measurement, and the like. “Determining” also means detecting or quantifying interaction between species, e.g. detection of binding between two species.

The term “self-assembled monolayer” (SAM) refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A wide variety of SAMs can be used in accordance with the invention, on a wide variety of surfaces, to present desired species such as binding partners, signaling entities, and the like at a surface of an article such as an electrode, intermediate, colloid particle, or the like. Those of ordinary skill in the art can select from among a wide variety of surfaces, functional groups, spacer moieties, etc. An exemplary description can be found in U.S. Pat. No. 5,620,850. See also Laibinis, P. E.; Hickman, J.; Wrighton, M. S.; Whitesides, G. M. Science 245, 845 (1989), Bain, C.; Evall, J.; Whitesides, G. M. J. Am. Chem. Soc. 111, 7155-7164 (1989), Bain, C.; Whitesides, G. M. J. Am. Chem. Soc. 111, 7164-7175 (1989), each of which is incorporated herein by reference.

The term “self-assembled mixed monolayer” refers to a heterogeneous self-assembled monolayer, that is, one made up of a relatively ordered assembly of at least two different molecules.

One aspect of the invention involves an immobilization relationship between a colloid particle and a non-colloidal structure which involves an intermediate entity. Assays involved in this aspect of the invention include steps such as allowing a colloid particle the ability to become immobilized indirectly relative to a non-colloidal structure, and determining immobilization of the colloid particle relative to the non-colloidal structure. Preferably, this involves allowing the colloid particle the ability to become immobilized relative to an intermediate entity which, in turn, has the ability to become immobilized relative to the non-colloidal structure. The intermediate entity, in preferred embodiments, is an intermediate colloid particle.

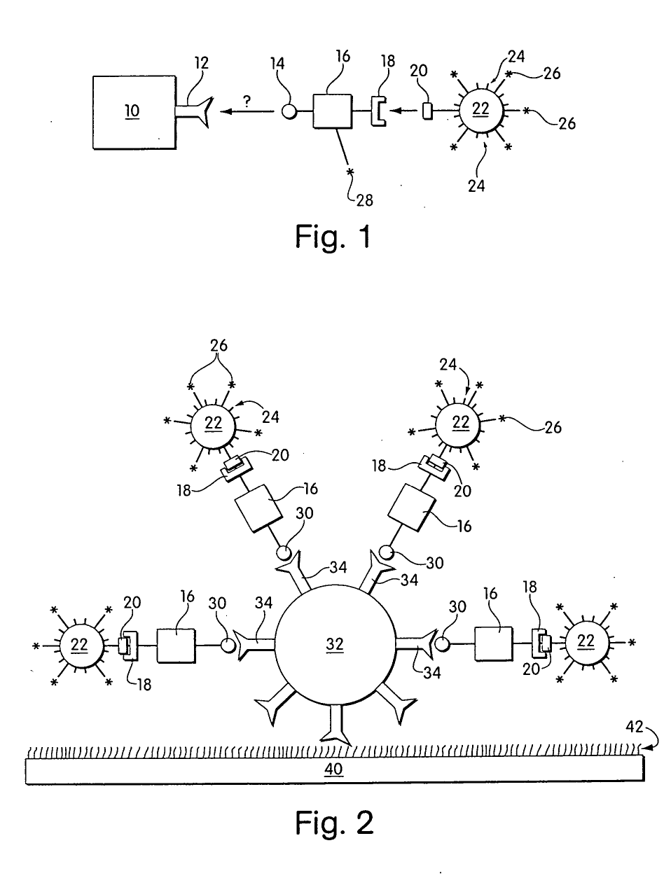

With reference to FIG. 1, one embodiment of the invention will be described. A non-colloidal structure 10 includes a portion 12 that is a putative binding partner of a species 14. Species 12 forms a part of, or is immobilized relative to, non-colloidal structure 10, and species 14 forms a part of or is immobilized relative to an intermediate entity 16. The intermediate entity includes a portion (or a species immobilized relative thereto) 18 which is a known binding partner of a species 20 that forms a part of or is immobilized relative to a colloid particle 22 which defines a signaling colloid. Colloid particle 22 includes a SAM 24 which includes one or more signaling entities 26. Intermediate 16 optionally includes one or more signaling entities 28. Signaling entities 26 and 28 can be precursors of signaling entities, or signaling entities themselves.

In a typical assay, intermediate 16 is first exposed to non-colloidal structure 10 for a period of time sufficient to allow species 12 and 14 to bind to each other if, indeed, they are binding partners. Whether they are binding partners or not can be the purpose of an assay. After a sufficient period of time, non-colloidal structure 10 and intermediate 16 are exposed to signaling colloid 22. Since species 18 and 20 are known binding partners, signaling colloid 22 will become immobilized relative to intermediate entity 16. If species 12 and 14 are binding partners, then signaling entity 26 is thereby brought into proximity of non-colloidal structure 10. Various reasons for using an arrangement as illustrated in FIG. 1 are described below.

One situation in which the tandem arrangement of the invention (FIG. 1) is particularly advantageous is one in which the colloid particle carries a species that could damage non-colloidal structures 10, e.g., kill the cell, for example a signaling entity (e.g., ferrocene). Ferrocene contains iron which can interact with iron receptors on a cell, causing the cell to engulf the colloid particle which can result in cell death. In the arrangement of the invention, an intermediate entity 16 such as a colloid particle that does not have the potential to damage the cell (non-colloidal structure 10) can first be exposed to the cell. The intermediate entity or colloid can carry a ligand 14 for a cell receptor 12 and, where a relatively long period of time (such as hours) may be required for ligand receptor interaction that binds the intermediate entity to the cell, this can be allowed to occur without damage to the cell. Then, where the intermediate entity exposes a binding partner 18 for the colloid particle, and the colloid particle carries a signaling entity 26 that potentially could damage the cell, exposure of the arrangement to the colloid particle will allow rapid binding of the colloid particle to the intermediate entity and signaling, without danger of cell damage or death, in the timeframe of the assay.

Another advantage to the system of FIG. 1 is that false positives in binding assays can be reduced. For example, in a typical prior art assay where binding between species 12 and 14 is to be determined, entity 16 functions not as an intermediate but as the signaling entity itself. In such a case, entity 16 is exposed to non-colloidal structure 10 and, if binding between species 12 and 14 occurs, then a signaling entity immobilized relative to entity 16 will be determined in proximity of structure 10. However, with the arrangement of FIG. 1, in which both binding between species 12 and 14 and binding between species and 18 and 20 is required to bring signaling entities 26 into proximity of structure 10, the number of false positives is reduced. Immobilization of species 20 and 26 to colloid 22, and immobilization of species 14, 18 and optionally 28 to intermediate 16 can be accomplished using any technique known in the art and/or described herein. Preferred embodiments involve affinity tag linkages and self-assembled monolayers as described in the above-referenced international publications of Bamdad, et al. For example, where intermediate. 16 is also a colloid particle, self-assembled monolayers can be formed upon intermediate 16 and colloid 22 including chelates coordinating metals (which can participate in metal binding tag/metal/chelate linkages). Then, species 14, 18, 28, 20, and 26 can be immobilized relative to their respective colloid particles by being modified to include a metal binding tag.

The non-colloidal structure can be any species such as cell, fluid-suspendable particle such as a bead including a magnetic bead, tissue specimen, polymer, dendrimer, hapten, plate (multi-well plate), electrode, or the like. The invention is not limited in any way by the identity of the non-colloidal structure. Any combination of non-colloidal structure 10 and signaling colloid 22 of the invention, whether signaling entities 26 do or do not affect non-colloidal structure 10, can benefit by a reduction in false positives.

Linkage between the colloid 22 and the intermediate entity 16, and between the intermediate entity and the non-colloidal structure 10, can include any chemical or biological linkage described herein, or known in the art. Preferred linkages include species fastened to self-assembled monolayer-forming species that in part define SAMs on the colloid particle, which can be binding partners for binding interactions between the colloid particle, intermediate entity, and the non-colloidal structure. Binding between any of these structures can be direct, such as between biological binding partners, or indirect, such as between Protein A or Protein G and antibodies. Binding between species 12 and 14, and between species 18 and 20 can be biological binding, chemical binding, biochemical binding, or a combination (e.g., a biological molecule binding to a chemical species). As noted, species 18 and 20 preferably are known to be strong binding partners (e.g., streptavidin/biotin, antibody/antigen, etc.). Species 12 and 14 typically are putative binding partners, i.e. the assay is useful in determining whether they indeed are binding partners.

The intermediate entity 16 can be any species suitable for performing the function describe, and can be readily selected by those of ordinary skill in the art. Entity 16 can include, inherently, binding species 14 and 18, or can be adapted for immobilization of species 14 and 18. Examples of entities 16 include a micelle, a liposome, a cell, a biological complex such as a proteinaceous complex (for example an antibody/antigen complex, a protein/DNA complex, a protein/small molecule complex), a dendrimer, a polymer, a drug, etc. Intermediate entity 16 also can be a fluid-suspendable particle such as a colloid, bead, etc. In one embodiment intermediate entity 16 is a colloid particle including a self-assembled monolayer, similar to colloid particle 22.

The colloid particle 22 can itself serve as a signaling entity in assays according to this aspect of the invention (i.e., signaling entity 26 may be optional), or the colloid, the intermediate entity, or both can include an auxiliary signaling entity. One feature of this aspect of the invention is close proximity between the colloid particle and the intermediate entity. This allows for signaling techniques involving communication between a first signal-involved entity 26 on the colloid particle and a second signal-involved entity 28 on the intermediate entity. One example involves a fluorescent signaling entity on the colloid particle or the intermediate entity, and a species having the ability to quench fluorescence of the fluorescent molecule on the other of the colloid particle or the intermediate entity. In a preferred arrangement structure 10 is first exposed to intermediate entity 16 carrying first signal-involved entity 28, and is added at a level sufficient to cover structure 10 to a high degree. For example, it can substantially completely coat the structure. Then, colloid particles 22 can be added carrying the second signal-involved entity 26 in an amount sufficient to bring a high enough number of the first and second signal-involved entities in proximity to each other to cause a high signal level. For example, where the intermediate entity 16 is an intermediate colloid particle carrying a fluorescent entity, and the colloid particle 22 carries a species able to quench fluorescence of the fluorescent entity, then where neither is present in sufficient quantity there can be a significant number of fluorescent entities that are not quenched. On the other hand, if the intermediate colloid particle is added in a high number, such as to substantially completely coat structure 10, and colloid particle 22 carrying a quenching moiety is added in a high number to substantially completely coat intermediate entity 16 on structure 10, then quenching can be substantially complete, and a change in signal is readily detected. Such arrangements can facilitate very distinct signaling in assays of the invention, and can further assure the absence of false positives in comparison to a typical assay simply involving a signaling entity becoming immobilized directly to structure 10.

The present invention can find use in interaction between chemical or biological agents for analysis, drug screening, or the like. The invention includes but is not limited to analyzing and/or inhibiting ligand interactions, including but not limited to ligands on intact cells (growing on an electrode, or in solution or in suspension). The present invention contemplates a variety of embodiments, including the use of drug candidates, known or putative ligands, and small molecule drug libraries.

In one embodiment, non-colloidal structure is a cell and the arrangement of the invention prevents damage to the cell by signaling entities 26 on signaling colloid 22. In such an arrangement, in other arrangements where signaling entities 26 are electroactive signaling entities, the signaling entities may be detectable using an electrode. In one example of such an arrangement, cells are grown on electrodes that may or may not be derivatized with self-assembled monolayers (SAMs). Putative ligands (e.g. for a particular cell-surface receptor) are immobilized on or form a part of an intermediate entity 16, and a signaling colloid 22 is also provided. These derivatized components are incubated with the cells immobilized on a sensing electrode (e.g. metal support). The interaction between the target receptor and the ligand on the solid support (e.g. intermediate-bound ligand) and the interaction between species 18 and 20 of intermediate 16 and signaling colloid 22, respectively, tethers the co-immobilized signaling elements near the sensing electrode. While not limited to any particular mechanisms, it is believed that, as a potential is applied to the electrode, the nearby redox-active metal complexes go through their characteristic oxidation potential and eject electrons. When an oscillating component is added on top of the voltage ramp, many electrons are ejected by each metal complex and can be detected as current output. A form of this sort of electrochemical analysis is called alternating current voltammetry (ACV).

As one example of a technique of the invention, with reference to FIG. 2, an example of a useful technique involving fastening of a signaling colloid particle to a cell is described. The tumor marker, MUC-1, is aberrantly expressed on neoplastic cells. The human tissue culture breast carcinoma cell line, MCF-7, available from the ATCC, over-expresses MUC-1. Antibody 30, DF3 or and DF3-p, available from the National Cancer Institute, is attached to (immobilized relative to) intermediate entity 16 which is known to be immobilizable relative to signaling colloids 22 via the known binding between species 18 and 20 (see also FIG. 1). Target cells 32 are incubated with the antibody-bearing intermediates for a period of time sufficient to allow antibody 30 to bind to the cell surface receptors 34, and then, following a wash step, are incubated with signaling colloids 22, then electrophoresed to an electrode 40 coated with a SAM 42 which can contain molecular wires, and analyzed by ACV. The SAM on electrode 40 can include molecular wires admixed within more conventional, tight-packing SAM-forming species. A current peak results if the antibody-bearing signaling colloids are incubated with cells bearing MUC-1.

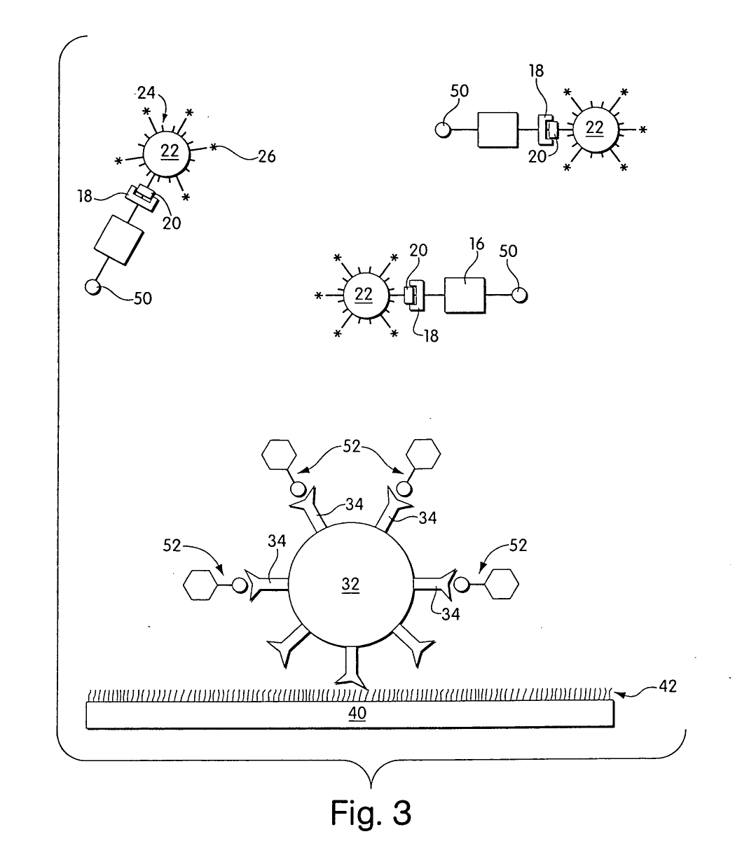

Another assay is shown in FIG. 3. Drug libraries can be screened for their ability to disrupt specific interactions with cell surface proteins, such as a MUC-1/Ligand interaction. The Ligand is bound to (or immobilized relative to) intermediate 16, then incubated with cells 32 presenting MUC-1 (34) and control cells. Drug candidates 52 are added to the solution within which the cells and intermediates are suspended, then the cells adhere to the electrode. Following sufficient time for the drug candidates to bind, or not to bind, to receptor 34, and following a wash step, signaling colloids 22 are introduced and (as described above) will rapidly become immobilized relative to intermediates 16 if intermediates 16 have become immobilized relative to cell 32. The system then is analyzed by ACV. A loss of signal indicates an interaction with a drug candidate. As illustrated, drug candidate 52 has blocked receptors 34, and signaling colloids 22 are immobilized relative to intermediates 16 and are suspended in solution remote from electrode 40 and do not produce a signal (of course, if intermediates 16 are washed away prior to introduction of signaling colloids 22, then colloids 22 and intermediates 16 will not become immobilized relative to each other as illustrated).

In another arrangement in which for a gain of signal assay, or to screen for drugs to bind cell surface receptors 34 for which the cognate ligand is not known, small molecule drug libraries can be synthesized on, or covalently attached to, intermediates 16. Drug candidates attached to intermediates can be incubated with cells presenting the receptor of interest, or control cells for a period of time sufficient to allow binding to take place, then the system can be exposed to signaling colloids 22. A drug-target interaction in this assay will result in a gain of signal.

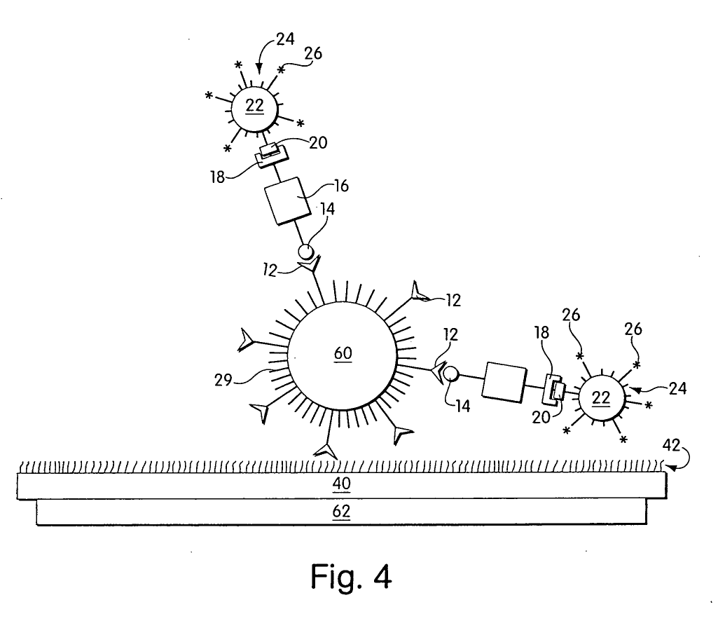

One embodiment of the invention involves using magnetic beads to recruit an electronic or electrochemical signaling entity to the surface of an electrode indicating capture of a binding moiety (a biological or chemical agent) by a binding partner. Referring to FIG. 4, a first species 14 is immobilized relative to intermediate 16 which, as described with reference to FIG. 1, can readily become immobilized relative to signaling colloid 22. A second species 12, which is suspected of being a binding partner of 14, is attached to a magnetic particle 60 that cannot signal, but can be magnetically attracted to an electrode 40. In one particularly useful technique species 14 and a second species 12 are proteins, thus the invention finds particular use in the field of proteomics. The technique is a example of an aspect of the invention involving allowing a colloid particle 22 the ability to become immobilized indirectly relative to a non-colloidal structure, magnetic bead, 60. Determining immobilization of the colloid particle relative to the non-colloidal structure is carried out by drawing the magnetic bead to the electrode (via magnet 62) and determining whether the colloid particle is also proximate the electrode, or unattached. Specifically, the magnetic particle and intermediate are incubated together in solution for a period of time to allow binding between species 12 and 14 (if, indeed, binding is to occur), and then, following a wash step, the system is incubated additionally with signaling colloid 22. Resultant complexes are magnetically attracted to the electrode. Electrodes are then analyzed by Alternating Current Voltammetry (ACV). (Laviron E: J Electro Anal Chem., 1979, 105: 35). Current is plotted, in real time, as a function of voltage. As noted, this arrangement is particularly useful where false positives are desirably minimized, and/or where signaling entity 26 and/or signaling colloid 22 is damaging in some way to any species associated with magnetic bead 60 (e.g., species 12), or even species 14 of intermediate 16.

In all embodiments involving electronic detection, if the electronic or electrochemical signaling moiety on the first component is brought very close to the electrode, a distinctive current peak will occur at a characteristic potential. If putative binding partners 12 and 14, or other putative binding partners interact with each other, then when the magnetic particle is attracted to the sensing electrode, it will also carry the colloidal particle, with the signaling capability, with it. In preferred embodiments where colloid 22 is fluid suspendable, e.g. is a small gold colloid, it will remain in suspension unless it is specifically recruited to the sensing electrode via binding interaction under study. In this embodiment, the colloid particle comprises an auxiliary signaling entity, exemplified by ferrocene. In other embodiments described below, the colloid particle is itself a signaling entity and no auxiliary entity is required.

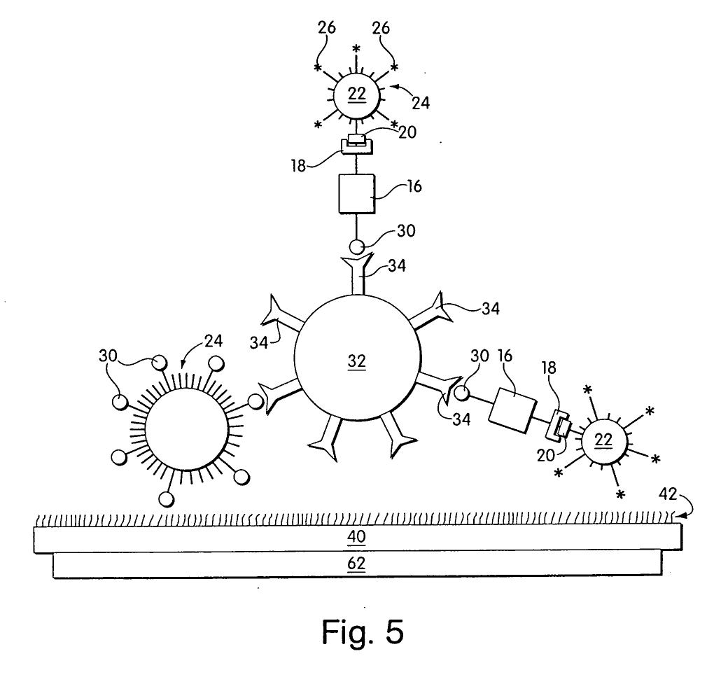

Referring now to FIG. 5, another arrangement of the invention is illustrated which is, in a sense, a combination of the arrangements of FIGS. 4 and 2. The arrangement of FIG. 5 can also be used in connection with drug screening or diagnostics as illustrated in FIG. 3. In FIG. 5, a magnetic particle 16 includes a self-assembled monolayer 24 incorporating a species 30 which is a putative binding partner of cell surface receptor 34 of a cell 32. Intermediates 16 also are provided including immobilized species 30 and species 18 suitable for rapid immobilization relative to signaling colloids 22, as described with reference to FIG. 1. Incubation of intermediate 16 with cell 32 and bead 60 will cause the intermediate, cell, and bead all to be immobilized relative to each other if binding between species 30 and receptor 34 takes place. Incubation is carried out for a period of time sufficient to allow such immobilization to occur (if, indeed, it does occur). Then, the system is exposed to signaling colloids 22 which will rapidly become immobilized relative to intermediates 16. Magnetically drawing magnetic beads 60 to electrode 40 will bring signaling entities 26 into proximity of the electrode if binding between species 30 and receptor 34 takes place. This proximity can be detected electronically as described above. Alternatively, beads 60 can first be incubated with cells 32 for a period of time sufficient to cause binding, and then magnetically drawn to the surface of electrode 40, followed by introduction of intermediates 16 which are allowed to bind, followed by introduction of signaling colloids 22. A wash step can take place following recruitment of beads 60 to the surface and prior to introduction of intermediates 16, or following introduction of intermediates 16, or both.

Certain embodiments of the invention make use of self-assembled monolayers (SAMs) on surfaces, such as surfaces of colloid particles, and articles such as colloid particles having surfaces coated with SAMs. In one set of preferred embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control. “Completely cover” in this context, means that there is no portion of the surface or region that directly contacts a protein, antibody, or other species that prevents complete, direct coverage with the SAM. I.e. in preferred embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently. A self-assembled monolayer on a surface, in accordance with the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species. The present invention provides a method for rigorously controlling the concentration of essentially any chemical or biological species presented on a colloid surface or any other surface. In many embodiments of the invention the self-assembled monolayer is formed on gold colloid particles.

The methods described in the present invention produce self-assembled monolayers on colloids that resist non-specific adsorption without protein blocking steps, such as blocking with BSA. The methods described herein also produce derivatized colloids that are stable in biologically relevant fluids and do not require detergents (for stability; maintaining colloids in suspension), which interfere with binding reactions. This allows sensitive binding assays to be performed in solution. This abrogates the need for having binding partners adhered to adsorbent surfaces, as is common for existing colloid agglutination assays. As is discussed below, detergent can advantageously be used for SAM formation on colloids. In this case, detergent can be and preferably is removed after SAM formation and is no longer present on the colloid, in the SAM, or elsewhere during binding interactions or other use of the colloids.

Certain embodiments described herein involve bringing cells in proximity of electrodes. A variety of techniques are contemplated for accomplishing this and are described as follows. In one set of embodiments, cells are bound directly to an electrode. In other embodiments, cells are indirectly bound through the interaction of ligands attached to the metal support. That is, cells can also be recruited to a surface by coating that surface with molecules that directly or indirectly bind to cells, by specific or nonspecific interactions. For example, methyl-terminated self-assembled monolayers (SAMs) bind collagen non-specifically, which in turn binds cells non-specifically. Alternatively, peptides that contain an argenine, glycine, aspartate (RGD) motifs, bind to many cell types, like endothelial cells. Similarly, polylysine, positive charge, Kringle domains, integrins, and peptide, or molecular mimics of the same, can be bound to surfaces for the attachment of cells. These ligands can be displayed on a surface by incorporation into SAMs. The ligands themselves need not be directly incorporated into the SAM. SAMs that display a binding partner for an affinity tag attached to the ligand may be used. For example, peptides modified with a histidine tag can be easily attached to SAMs that contain a nitrilo tri-acetic acid (NTA)—nickel thiol. Alternatively, glutathione S-transferase fusion proteins can be attached to a SAM that incorporates glutathione or a derivative thereof. One advantage of using a metal electrode is that many known functional groups can be provided at the terminus of molecules that will form a SAM on the surface.

Cells in solution, for example, can be attracted to a detecting electrode by electrophoresis. Specifically, cell-derived molecules can be bound to a ligand(s) that are attached to a colloid that also displays electro-active compounds such as ferrocene derivatives to aid in the detection of the bound complex. However, the detection element used can be any charged, electro-active species or fluorescent tag that can be easily detected or the colloid itself.

The electrode surface used can take various forms. However, for illustrative purposes, methods for recruiting a binding partner complex are described that use an electrode that is modified with a conducting SAM. A conducting SAM is a layer of molecules attached to a metal surface that allows the conduction of electrons at a rate that is higher than a metal uniformly coated with an insulating species such as saturated alkyl thiolates. A preferred pathway for electron conduction can be provided by a monolayer into which molecular wires have been incorporated.

Cells can be recruited to an electrode coated with a conducting SAM that also presents molecules that are terminated with head groups that directly or indirectly bind to cells (e.g. methyl groups, poly K, positive charge, RGD sequences, Kringle motifs, integrins, and peptide mimics of the same).

Cells also can be attracted to the electrode due to the fact that they are negatively charged when a slight positive bias to the AC voltage ramp is applied. Adding additional negatively charged groups to the dendrimers or polymers to which the ligands are attached further facilitates recruitment to the sensing surface. Also, sensing electrodes that incorporate ligands, either directly or through a histindine tag, can be used to attract cells via specific interactions, such as with cell adhesion molecules or non specific interactions.

Recruitment of cells to the detection system (electrode) can be performed also simply by using gravity if the complex sediments by gravity as a result of one of the binding partners being denser than the analysis solution. Mechanical mixing can be performed during the incubation stage to avoid premature sedimentation.

In one specific embodiment, one can detect and quantitate cell surface proteins as follows: Histidine-tagged ligands that recognize cell surface receptors are attached to intermediates that can be linked to colloids (FIG. 1) that bear SAMs presenting both NTA (to capture His-tagged proteins) and ferrocene moieties (for electronic or electrochemical signaling). These biospecific intermediates, followed by electronic or electrochemical signaling colloids are then incubated with cells presenting target receptors. Cells are then allowed to sediment, adhere, or be attracted onto to a SAM-coated electrode and analyzed by ACV. A current peak, at the ferrocene moiety's characteristic oxidation potential, will result if ligands immobilized on intermediate entities bound to their cognate receptors on the cell surface. Antibodies that recognize the cell surface receptor can be attached to NTA-ferrocene bearing intermediates that have first been bound with His-tagged protein A or G. Alternatively, an antibody can be attached directly to an intermediate via a metal binding tag/metal/chelate linkage, where the metal binding tag is linked to the antibody. Techniques for linking a histidine tag to an antibody can be found in “Construction of the single-chain Fv from 196-14 antibody toward ovarian cancer-associated antigen CA125” Hashimoto, Y., Tanigawa, K., Nakashima, M., Sonoda, K., Ueda, T., Watanabe, T., and Imoto, T.: 1999, Biological and Pharmaceutical Bulletin, Vol 22: (10) 1068-1072.; “Human antibodies with sub-nanomolar affinities isolated from a large non-immunized phage display library”, Vaughan, T. J., Williams, A. J., Pritchard, K., Osbourn, J. K., Pope, A. R., Earnshaw, J. C. et al. 1996, Nature Biotechnology Vol 14 (3) p. 267.; “Expression and purification of single chain anti-HBx antibody in E. coli” Zhou G, lui K D, Sun H. C., Chen Y. H., Tang Z. Y., and Schroder C. H., 1997, vol. 123(11-12) pgs 609-13.

The following is a description of one specific assay that can be assisted by the arrangement of the invention.

The cell surface receptor, αVβ3, has been implicated in promoting angiogenesis through an interaction with a cell adhesion molecule vitronectin. Human umbilical veinous endothelial cells (HUVEC) that present αVβ3 cell surface receptors are commercially available from Clonetics. To screen for drugs that inhibit the action of αVβ3, His-tagged peptides that present RGD-containing sequences, derived from vitronectin, are attached to intermediate colloids 16 that bear SAMs presenting NTA groups. The intermediates are then incubated with HUVEC cells and drug candidates for a period of time sufficient to allow binding to occur, followed by a washed step, then incubation with signaling colloids 22 (with binding capability relative to intermediate 16 as shown in FIG. 1). The HUVEC cells can be grown on the electrode. However, if the cells are in solution or suspension they can be electrophoresed or magnetically attracted to a sensing electrode and analyzed by ACV. A current peak occurs when signaling colloids and intermediates are incubated with HUVEC cells, rather than control cells. If a drug candidate interferes with the αVβ3-RGD sequence interaction, a loss of signal results. The assay can be conducted electronically, as discussed, or visually by growing the cells in a standard plastic dish, conducting the assay, and viewing the cells under 40-fold magnification (in which case, of course, electronic signaling entities 26 need not be used). It is to be understood that in all embodiments of the invention signaling colloids 22 can be used with or without auxiliary signaling entities 26, and signaling entities 26 can be any signaling entity described herein. Appropriate signaling entities will be selected easily by those of ordinary skill in the art based upon a particular assay configuration.

Alternatively, drug candidates can be synthesized on, or attached to, intermediate entities 16 and used in conjunction with signaling colloids 22 as shown in FIG. 1. In such an arrangement, components are incubated with target cells, attracted to a sensing electrode and analyzed by ACV. The attractive field is then reversed and peptides containing RGD sequences are titrated into the solutions. The cells are again electrophoresed to the sensing electrode and re-analyzed. A loss of signal indicates that the drug-cell interaction is specific for the αVβ3 receptor and the IC₅₀ of the RGD peptide can be correlated to a binding affinity for the drug.

Alternatively, a 49 amino acid peptide, echistatin, that also binds to αVβ3, can be His-tagged to replace the RGD-containing peptide in the above-described assay.

Cell-surface molecules can be detected on cells in suspension or embedded in a tissue sample. In such an arrangement the following would take place: Frozen tumor specimens are cryo-sectioned and placed directly onto a flexible, semi-permeable membrane support that has been derivatized with cell-binding groups such as RGD-containing peptides or methyl-terminated groups. The specimen is then incubated first with intermediate entities 16 that present ligands for a cell surface receptor of interest and, following a sufficient period of time for binding to occur, and a wash step, signaling colloids 22 that can bind to intermediates 16 as shown in FIG. 1. The support membrane is then placed in physical contact with a microelectrode array, having electrode dimensions comparable to cell size, and analyzed by ACV. Each sector of the tissue specimen is analyzed for protein content and expression level, then correlated with histopathology. This capability ensures the relevance of single cell analysis because it enables the researcher to identify protein patterns that are associated specifically with cancer cells and discard random aberrant protein expression. Cells in suspension can be similarly attached to the support membrane.

This technique can be used to identify cell-derived molecules, such as receptors or proteins, that are expressed differentially in healthy versus diseased tissue or cells. This differential expression can involve different levels of an expression in healthy versus diseased tissue or cells, and/or different patterns of expression on tissues or cells which can be readily identified. This technique facilitates diagnostic assays for determination of disease states. For example, in connection with a patient suspected of having a particular disease, cells can be taken from the patient, specifically, cells that are associated with an indicator of the disease such as cells from a biopsy, blood sample, etc., and these cells can be analyzed versus healthy cells to determine expression levels or patterns indicative of disease.

The invention also provides the ability to visually investigate patterns of cell surface receptor expression on individual cell surfaces and/or on cells embedded in a tissue specimen. This can be indicative of the pattern of cell surface receptor expression which can be correlated to a disease state. These can also be used in diagnostics or drug screening methods. In a particular assay, colloid particles carrying ligands that bind to cell surface receptors are exposed to individual cells or embedded cells and the location of their binding with respect to individual cells can be determined visually, indicating the pattern of cell surface receptor expression. Visual identification, in this embodiment, can involve any technique described herein such as observation with the unaided human eye, microscopy, spectrophotometry, electron microscopy, fluorescence detection, etc.

In the technique involving electronic or electrochemical detection described above, the levels of expressed species can be compared between samples, including samples each involving an individual cell or other very small quantity, and patterns can be determined on larger samples including tissue samples. In connection with the visual detection embodiment described above, levels of expressed species can be determined as well as patterns of expressed species on both large samples and small samples including single-cell samples. Multiple signaling entities can be used (i.e., multiple signaling per binding event). In connection with both electronic or electrochemical or visual signaling, different signaling entities can be used in connection with different assays. For example, a first ligand selected to target a first receptor or protein may be immobilized with respect to a first intermediate uniquely able to bind to a first signaling entity while a second ligand, selected to target a second protein or receptor can be immobilized with respect to a second intermediate uniquely able to bind to a second signaling entity. In electronic or electrochemical signaling the different signaling entities can include different redox potentials, the difference between which is distinguishable electronically, and in connection with visual identification different signaling entities can be different colors of emissive or absorptive entities. In such a case not only can expression level and pattern of proteins or receptors be determined but patterns can be differentiated in terms of location of expression of one receptor or protein versus another.

The function and advantage of these and other embodiments of the present invention will be more fully understood from the examples below. The following examples are intended to illustrate the benefits of the present invention, but do not exemplify the full scope of the invention. For example, with reference to FIG. 1, the linkage between intermediate 16 and non-colloidal structure 10, and between colloid 22 and intermediate 16, need not be precisely as shown. Other arrangements can be used that allow, first, binding of species 14 to species 12, followed by any series of interactions necessary to immobilize signaling colloid 22 relative to species 14 if species 14 has bound to species 12. In one example, species 14 is first allowed to bind to species 12 without the presence of intermediate 16. Then, intermediate 16 is allowed to bind to species 14 and colloid 22 is allowed to become immobilized relative to intermediate 16. In such a case, species 14 can be considered the intermediate entity itself, and the colloid particle has the known ability to become immobilized relative to the intermediate entity (14 according to this example), via its ability to become immobilized relative to intermediate 16, which, in turn, has the ability to become immobilized relative to species 14. In another example of modification that falls within the scope of the invention, species 18 and 20 need not necessarily be binding partners. It is important only that signaling colloid 22 has the known ability to become immobilized relative to intermediate 16. For example, species 18 and 20 can each comprise biotin, and can be linked to each other via streptavidin, as would be known by those skilled of ordinary skill in the art.

The following examples are prophetic examples, describing how one would conduct these procedures.

### EXAMPLE

### General Procedures

For SAM formation, glass microscope slides are sputtered with a layer of Ti followed by a layer of Au. Each electrode is incubated at room temperature for 0.5 hours with 300 uL of a DMF solution that contains 10% methyl-terminated thiol (HS—(CH2)15 CH3), 40% tri-ethylene glycol-terminated thiol, HS(CH₂)₁₁(CH₂CH₂)₃0H, (formula) and 50% MF-1. 2 ml of 400 uM tri-ethylene glycol-terminated thiol are then added to a scintillation vial containing the chip and the vial is heat cycled in a water bath as follows: 2 minutes @ 55° C.; 2 minutes @ 37° C.; 1 minute @ 55° C.; 2 minutes @ 3720 C. then RT for 10 min. Electrodes are then dipped in EtOH, then sterile PBS to rinse. They are then placed under the LTV germicidal lights in a biosafety cabinet for I hour to ensure sterility.

For collagen coating, a 200 uL droplet of 0.005 mg/ml collagen in PBS is added to each electrode and incubated at 4° C. for 2 hours.

For cell growth, the electrodes are placed in a cell growth flask and a solution of growth media and human endothelial cells (HUVECs), presenting a particular cell surface receptor, αVβ3, is added. The electrodes and cell containing solution are incubated at 37° C. in a C0₂ incubator for 24 hours.

For colloid preparation, 1.5 ml of commercially available gold colloid (Auro Dye) are pelleted by centrifugation in a microfuge on high for 10 minutes. The pellet is resuspended in 100 uL of the storage buffer (sodium citrate and tween-20). 100 uL of a dimethyl formamide (DMF) solution containing 90 uM nitrilo tri-acetic acid (NTA)-thiol, 90 uM ferrocene-thiol, and 500 uM carboxy-terminated thiol. Following a 3-hour incubation in the thiol solution, the colloids are pelleted and the supernatant discarded. They are then incubated in 100 uL of 400 uM tri-ethylene glycol-terminated thiol in DMF for 2 minutes at 55° C., 2 minutes at 37° C., 1 minute at 55° C., 2 minutes at 37° C., then room temperature for 10 minutes. The colloids are then pelleted and 100 ul of phosphate buffered saline (PBS) is added. The colloids are then diluted 1:1 with 180 uM NiS04 in the colloid storage buffer. 100 uL of a His-tagged peptide at 100 uM in PBS is added to 100 uL of NTA-Ni(II) presenting colloids and incubated for 0.5 hours. To get rid of free, unattached peptide, the colloids are then pelleted and the supernatant discarded. The colloid pellet is then resuspended in 100 uL PBS. Colloids are bound with either: a) a peptide designed to bind to the αVβ3 receptor, HHHHHH(S₄G₁)₃GRGDSGRGDS (SEQ ID NO: 1); or b) an irrelevant peptide, HHHHHH (SEQ ID NO: 2)-Glutathione S-Transferase (GST). Peptides containing an RGD motif have been shown to bind to the αVβ3 receptor on endothelial cells. It is thought that RGD motifs in vitronectin (the natural ligand for αVβ3) are responsible for the interaction.

ACV Analysis is performed using a CH Instruments electrochemical analyzer. A three-electrode system is used. A silver vs. silver chloride reference electrode is used with a platinum auxiliary electrode. The derivatized gold-coated chip is used as the working electrode. A 25 mVolt overpotential is applied to the electrode at a frequency of 10 Hz.

### Example 1

In this example, cells are attached to gold-coated electrodes derivatized with SAMs. The cells, which are still attached to the electrode, are then incubated with a solution containing intermediates which had been derivatized to present a ligand specific for a receptor on the cell surface, and then incubated with a solution containing signaling colloids immobilized with a redox-active metal capable of delivering an electronic or electrochemical signal to the electrode. After some incubation period, the electrodes are scanned by alternating current voltammetry (ACV). A positive interaction between the ligand and the cell surface receptor will bring the redox-active metal, on the colloid, close enough to the electrode to transduce an electronic or electrochemical signal.

More specifically, electrodes are derivatized with SAMs to present 10% methyl head groups in a background of 50% Bis(ethynylphenyl thiol) (i.e. C₁₆H₁₀S) to facilitate electron flow to the electrode. 40% triethylene glycol-terminated thiols (HS(CH)₂)₁₁(CH₂CH₂)₃OH) are included to help monolayer packing. It had previously been shown that cell growth can be supported on HSC15-methyl-terminated SAMs that were coated with collagen. Both the HSCH₂C₁₅CH₃ and the collagen are insulating molecules and can inhibit electron flow to the electrode. For this reason, in this example, the saturated carbon chain-collagen coverage is reduced to produce islands of growing cells adjacent to the more conducting molecular wires. Human endothelial cells (HUVECs), that present a cell surface receptor αVβ3, which is important for angiogenesis, are grown on the electrodes. SAM-coated intermediate gold colloids bearing a ligand for the receptors and signaling colloids carrying ferrocene moieties for electronic or electrochemical signaling are briefly incubated with the cell-presenting electrodes, then analyzed by ACV.

For ACV analysis, a 1 ml capacity silicone gasket is clamped onto the cell-presenting electrode. 100 ul of NTA-Ni colloids that had been pre-bound with a His-tagged RGD motif peptide and 100 ul PBS is added to the gasket for incubation with the cell-presenting electrode. After 15 minutes, the first ACV scan is taken. Two successive scans are taken at 15 minute intervals. Current output is plotted against voltage. One scan may produce a broad current bulge characteristic of PBS buffer. Other scans generate distinctive current peaks at a characteristic ferrocene potential.

### Example 2

### Cell Growth on Conducting Surfaces

This example describes the electronic detection of cells grown on “conducting” surfaces that are not coated with collagen. Cells are grown on gold electrodes that are modified with sulfur-containing molecules, in some cases assembled into monolayers, but not coated with collagen. Electrode modification is performed as described in the electrode preparation section of Example 1, with the exception that electrodes incubated with 100% candidate molecule are not heat cycled in tri-ethylene glycol-terminated thiol. Several electrodes are assembled in the same cell growth flask and media containing HUVEC cells is added. The electrodes and cells are incubated in a C0₂ incubator for 24 hours. Surfaces are visually analyzed using 100× magnification. Cells can easily be immobilized on a SAM by presenting a metal chelate coordinating a metal via the SAM, linking a protein to the SAM via a metal binding tag on the protein, the protein attracting the cell. Cells are incubated with colloids (as described above) that display ferrocene moieties and a peptide, HHHHHH (S₄G₁)₃GRGDSGRGDS (SEQ ID NO: 1), that is specific for the αVβ3 receptor on the cell surface; or as a negative control, an irrelevant peptide, HHHHHH (SEQ ID NO: 2)-Glutathione S-Transferase (GST). Cells grown on a 100% ethynylphenyl thiol (MF1) SAM-coated electrode will produce current peaks only if incubated with colloids bearing the ligand specific for the αVβ3 receptor and not when incubated with colloids derivatized with an irrelevant peptide GST.

### Example 3

### Drug Screen

An example essentially identical to those described above is carried out in the presence of a drug candidate for binding to the cell surface receptor. If the drug candidate is effective, then no signal is detected at the electrode.

### Example 4

### Detection of Protein-Protein Interactions

This example demonstrates the utility of an intermediate of the invention in a non-cell Study (see FIG. 4)

Histidine-tagged Glutathione-S-Transferase (GST-His) is attached to NTA-SAM-coated intermediate colloids, displaying 40 uM NTA-Ni. Signaling colloids are similarly provided and carry 100 uM ferrocene-thiol. Commercially available magnetic beads presenting protein A are coated at 1/10 binding capacity with anti-GST antibody, added at a 1:5 ratio to the GST-colloids (intermediates) then exposed to the signaling colloids, and measured on a 50% MF-1 SAM-coated electrode, which is placed on top of a magnet. The magnet pulls the magnetic beads onto the electrode surface to form a thick, visible precipitate. The GST-colloids are brought down to the electrode surface by the interaction with the GST-antibody on the magnetic beads to give a current peak at approximately 280 mV. Two negative controls are run, one where GST is not attached to the colloid surface, and another where the GST antibody was not attached to the magnetic beads. Neither negative control gives a current peak.

### Example 5

### Cell Detection

This example demonstrates both the advantage of forming a SAM on a surface that includes a mixture including a molecular species that enhances electronic communication across the SAM by forming a defect in the SAM allowing fluid to which the surface is exposed to communicate electrically with the surface, and the utility of attachment of a colloid carrying immobilized signaling entity to a protein. The protein is in turn immobilized at a cell attached to the surface of an electrode presenting the SAM. The defect in this case is caused by bulk of the a SAM-incorporated molecule including phenyl rings.

HUVEC cells are suspended in media and placed in a flask over a SAM coated on a gold surface. The SAM includes 50% straight chain thiols, and 50% of the 2-unit poly (ethynylphenyl) thiol (MF1). 5 ul of an 8.4 mM RGD-His peptide solution is added to the media, and cells are incubated at 37° C. overnight to adhere to the electrode surfaces. After approximately 16 hours, 100 ul of SAM-coated intermediate colloids, displaying NTA for capturing the RGD-His peptide, and signaling colloids similarly prepared but bearing ferrocene for signaling, are added to the cells and incubated for 20 min at room temperature. The intermediate colloids are added first and allowed to incubate and, after sufficient time, the signaling colloids are added. The electrodes are then rinsed in buffer to wash off any unbound colloids and measured. Current peaks are recorded at 220-250 mV. Negative controls are cells incubated with His-GST, an irrelevant protein that should not bind to cells. Colloids are added to negative controls, electrodes are rinsed in buffer, and measurements are taken. No peaks are observed for negative controls.

### Example 6

### Attachment of Unmodified Chemical or Biological Molecules to Self-Assembled Monolayers Presented on Surfaces of Colloids, Specifically, Attachment of Streptavidin to Colloids

This example provides a technique for the attachment of essentially any amine-containing entity to a surface able to present a carboxylic acid or salt thereof. The technique can be used to attach the entity to a colloid, via attachment to a self-assembled monolayer formed on a surface of a colloid. The molecule that is attached to the self-assembled monolayer need not include any particular chemical functionality, such as an affinity tag or the like, prior to attachment. The only requirement is that the molecule include at least one primary amine, such as are found in many amino acids. Accordingly, the technique is particularly well-suited for the covalent attachment of proteins or peptides that carry primary amino groups to colloids that present carboxylic acids at their surfaces.

Techniques for formation of self-assembled monolayers on colloids are described above. These techniques were followed, with the following specific details and provisions.

A protein is attached by forming an amide bond between a carboxylate on a colloid surface (attached via a SAM) and an amine residue of the peptide. The amide bond is formed by the application of a modified EDC/NHS coupling protocol. The success of the coupling is tested by mixing colloids presenting streptavidin with colloids presenting biotin groups (via a biotin SAM, described previously). If the streptavidin is successfully attached then the two types of colloids will aggregate and the suspension will change color and, ultimately, the colloids will precipitate out leaving a clear solution.

The colloids used for the coupling procedure are prepared as described elsewhere and are formed by incubation in a thiol solution containing 540 uM (micromolar) COOH-terminated thiol, 60 uM NTA-terminated thiol, and then heat cycling in 400 uM ethylene glycol-terminated thiol. 50 ul of colloids in PBS are treated with 50 ul of 10 mM EDC, 40 mM NHS in water. After 7 min the colloids are spun down, the liquid is removed, and the colloids are resuspended in 500 ul (microliter) of 0.1 mg/ml streptavidin in phosphate buffer, pH 6.4. After 1.5 h, unreacted sites on the colloids are blocked by adding ethanolamine and the colloids are spun down, then resuspended in 100 microL of pH 6.4 phosphate buffer to wash away excess, uncoupled streptavidin. Colloids are then spun down again and resuspended in 50 ul PBS.

To a mixture of 20 microL of phosphate buffer and 15 ul of biotin-presenting colloids is added 15 ul of streptavidin-presenting colloids prepared above. This suspension is compared to a mixture of 35 ul of phosphate buffer and 15 ul of biotin-presenting colloids. The addition of streptavidin-presenting colloids causes an immediate change in color from red to blue. After ˜10 min the mixture of biotin colloids and streptavidin colloids appears clear with precipitate on the bottom. The mixture that did not contain the biotin colloids remains red with no visible change.

The above-described protocol is applicable to attachment of essentially any chemical or biological molecule including an amine to a self-assembled monolayer on a colloid. To ensure binding of a molecule to only one colloid, rather than binding to multiple colloids which could result in aggregation, the following adjustments can be made if appropriate. The concentration of colloid in solution can be decreased, while maintaining concentration of molecule desirably attached and maintaining concentration of EDC/NHS reactant. Other slight modifications can be made by those of ordinary skill in the art for various molecules. Amine-containing molecules can be selected by those of ordinary skill in the art and include, without limitation, proteins, synthetic molecules, peptides, dervatized nuclic acids, and other dervatized or naturally-occurring biological molecules that contain amines. As would be appreciated by those of ordinary skill in the art, a wide variety of species can be synthezised with or attached to amines or amine-containing species to render them attachable to a colloid according to this embodiment of the invention.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described.

1. A method comprising:
allowing a colloid particle to become immobilized indirectly relative to a non-colloidal structure; and
determining immobilization of the colloid particle relative to the non-colloidal structure.

2.-43. (canceled)
